(19) United States
(12) Patent Application Publication
(10) Pub. No.: US 2009/0326396 A1
(43) Pub. Date: Dec. 31, 2009

(54) DUAL BODY PLETHYSMOGRAPHY APPARATUS AND PROCESS FOR MEASURING BLOOD FLOW BETWEEN THE THORAX AND ABDOMEN (THE TRUNK) AND THE BODY PERIPHERY

(76) Inventors: Andrea Aliverti, Como (IT); Raffaele Dellacà, Como (IT); Antonio Pedotti, Milano (IT); Peter T. Macklem, Lansdowne (CA)

Correspondence Address: WENDEROTH, LIND & PONACK, L.L.P., 1030 15th Street, N.W.,, Suite 400 East, Washington, DC, 20005-1503, US

(21) Appl. No.: 12/309,757

(22) PCT Filed: Jul. 31, 2007
(86) PCT No.: PCT/EP2007/057919

§ 371 Date: Jan. 29, 2009

(30) Foreign Application Priority Data
Aug. 2, 2006 (IT) MI 2006A 001540

(51) Int. Cl. A61B 5/02

(52) U.S. Cl. 600/507

(57) ABSTRACT

An apparatus is described (1) for measuring the blood flow (Vb) between the trunk and the extremities. Such an apparatus (1) comprises a whole body plethysmograph (102; 4, 12, 15, 40) for measuring the variations of gas volume in the lungs (ΔVL), a thoraco-abdominal plethysmograph (101, 2, 3) for measuring the volume variations of the trunk (ΔVCW) and a system for processing (20) allowing measurement of the said variations of gas volume of the lungs (ΔVL) and the volume variations of the trunk (ΔVCW) so as to compute the volume of blood shifted between the trunk and the extremities (Vb). Further a process is described for measuring the volume of blood shifted between the trunk and the extremities (Vb). Such a process comprises: a measurement (121) of the volume variations of the trunk (ΔVCW) by means of thoraco-abdominal plethysmography, a measurement (104) (performed at the same time as the previous measurement) of the variations of gas volume of the lungs (ΔVL) by means of whole body plethysmography; a processing (120) of said volume variations of the trunk (ΔVCW) and of said volume variations of the lungs in order to measure the volume of blood shifted between the trunk and the extremities (Vb).

The present invention relates to a dual body plethysmography apparatus and process for measuring the blood flow between the trunk and the body periphery.

The measurement of the blood flow between the trunk and the body periphery (term by which the upper limbs, the lower limbs and the head are designated) is extremely important in the understanding of the physiology and physiopathology of cardiopulmonary interaction.

Changes of the pleural and abdominal pressure have important implications for cerebral blood flow, pulmonary blood volume, cardiac output and ventricular afterload. Changes in these parameters affect alveolar gas exchange and skeletal muscle perfusion particularly during physical exercise with or without limitation of expiratory flow as frequently occurs in chronic obstructive lung disease and asthma. These parameters are also affected by positive pressure mechanical ventilation, negative inspiratory pressures during obstruction of the upper respiratory tract (for instance in obstructive sleep apnoea), strongly positive expiratory pressures as occurs in Valsalva's maneuver, expulsive maneuvers, and cough.

Some of the aforesaid implications have been described in the following papers:
Aliverti A., Macklem P. T., “How and why exercise is impaired in COPD”, Respiration. 68: 229:239, 2001;
Aliverti A., R. L. Dellacà, P. Lotti, S. Bertini, R. Duranti, G. Scano, J. Heyman, A. Lo Mauro, A. Pedotti, P. T. Macklem. “Influence of expiratory flow-limitation during exercise on systemic oxygen delivery in humans”. European Journal of Applied Physiology, 2005;
Duke G. J. “Cardiovascular effects of mechanical ventilation”. Critical Care and Resuscitation 1999; 1: 388-399;
H. Fessler, S. Permutt. “Interaction between the circulatory and ventilatory pumps”. Thorax: 57, 1995;
Iandelli I, Aliverti A, Kayser B, Dellacà R, Cala S J, Duranti R, Kelly S, Scano G, Sliwinski P, Yan S, Macklem P T, Pedotti A. “Determinants of exercise performance in normal men with externally imposed expiratory flow limitation”. J Appl Physiol 92: 1943-1952, 2002;
Lloyd. C. Mechanical cardiopulmonary interdependence. “J. Appl. Physiol.: Respirat. Environ”. Exercise Physiol. 52: 333-339, 1982;
Lu, K., J. W. Clark, Jr., F. H. Ghorbel, D. L. Ware, and A. Bidani. “A human cardiopulmonary system model applied to the analysis of the Valsalva maneuver”. Am J Physiol Heart Circ Physiol 281: H2661-H2679, 2001;
Miller J. D., Pegelow D. F., Jacques A. J. and Dempsey J. “Effects of augmented respiratory muscle pressure production on locomotor limbs venous return during calf contraction exercise”. Articles in Pres. J Appl Physiol, 2005b;
Permutt S., A. Wise. “Mechanical interaction of respiration and circulation”. Handbook of physiology: the respiratory system III: 36, 1986;
Ranieri V. M., M. Dambrosio, N. Brienza. “Intrinsic PEEP and cardiopulmonary interaction in patients with COPD and acute ventilatory failure”. Eur Respir J, 9:1283-1292, 1996;
Scharf, S. M., J. A. Bianco, D. E. Tow, and R. Brown. “The effects of large negative intrathoracic pressure on left ventricular function in patients with coronary artery disease”. Circulation 63: 871-875, 1981;
Scharf, S. M., R. Brown, N. Saunders, and L. H. Green. “Effects of normal and loaded spontaneous inspiration on cardiovascular function”. J. Appl. Physiol.: Respirat. Environ. Exercise Physiol. 47: 582-590, 1979;
Takata, M., Wise, R. A., and Robotham, J. L. “Effects of abdominal pressure on venous return: abdominal vascular zone conditions”. J. Appl. Physiol., 69(6): 1961-1972, 1990.

There are currently no devices or processes which can measure blood shifts between the trunk and the extremities.

It is known that, during every respiratory act or maneuver, the variations of the volume defined by the thoraco-abdominal wall (hereafter also indicated by the symbol ΔV_{cw}, with reference to the term “chest wall”) are determined by the sum of the variations of gas volume in the lung (hereafter also indicated by the symbol ΔVL) and variations of blood volume contained within the trunk. (V_{b}) according to the equation

ΔV_{cw}=ΔVL+V_{b}.

There are known systems to measure ΔVL, by total body plethysmographies or by the acronym WBP, “Whole Body Plethysmography”, described in U.S. Pat. No. 3,511,237) and consisting in measuring the variations of air flow or volume in a constant pressure booth in which a subject under examination is seated; furthermore, the subject breathes through a mouthpiece which is connected to the outside; finally, there is an opening provided with a special sensor that measures the ΔVL. Two embodiments of such plethysmographies are considered here:
volume plethysmographs: the volume of input/output air to/from the booth is measured; see Mead J. “Volume displacement body plethysmograph for respiratory measurements in human subjects”. J Appl Physiol 1960; 15: 736-740;
flow plethysmographs: the flow of input/output air to/from the booth is measured; such a flow is then integrated in order to obtain ΔVL;

There are also known systems for the measurement of the variations of the volume of the trunk (ΔV_{cw}), known as thoraco-abdominal plethysmographs or by the acronym TAP, “Thoraco-Abdominal Plethysmography”.

A first example of thoraco-abdominal plethysmography is given by the opto-electronic plethysmography (also known by the acronym OEP, “Opto-Electronic Plethysmography”), described in patent ITMI001188A1 and by the papers:
Ferrigno G, Carnevali P, Aliverti A, Molteni F, Beulke G and Pedotti A. “Three-dimensional Optical Analysis of Chest Wall Motion”. J Appl Physiol 77(3): 1224-1231, 1994;
Cala S J, Kenyon C, Ferrigno G, Camevali P, Aliverti A, Pedotti A, Macklem P T and Rochester D F. “Chest wall and lung volume estimation by optical reflectance motion analysis”. J Appl Physiol 81(6): 2680-2689, 1996;
Carnevali P, Ferrigno G, Aliverti A and Pedotti A. “A new method for 3D optical analysis of chest wall motion”. Technology and Health Care 4: 43-65, 1996;
Aliverti A, R. Dellacà, P. Pelosi, D. Chiumello, A. Pedotti, L. Gattinoni. “Opto-electronic plethysmography in intensive care patients”. Am. J. Respir. Crit. Care Med., 161: 1546-1552, 2000;
Aliverti A, R. Dellacà, P. Pelosi, D. Chiumello, L. Gattinoni and A. Pedotti. “Compartmental analysis of breathing in the supine and prone positions by Opto-Electronic Plethysmography”. Ann Biomed Eng 29: 60-70, 2001;
Aliverti A, Pedotti A. “Opto-electronic Plethysmography”. Monaldi Arch Chest Dis; 59(1): 12-6, 2003.

Said opto-electronic plethysmography uses digital video cameras to film a subject under examination; reflective markers are applied on the chest and abdomen of such a subject. The markers are filmed by the video cameras, which transmit the filmed sequences to a microprocessor system, which traces the position in space of the markers, and computes the variations of the volume enclosed in the trunk (V_{cw}) on the basis of a pre-established geometrical model.

A second example of thoraco-abdominal plethysmography is given by the respiratory inductance plethysmography (also known by the acronym RIP), described by U.S. Pat. Nos. 5,331,968, 3,731,184, 4,308,872, 4,373,534, 4,433,693, 4,452,252, 4,494,553, 4,807,640, 4,815,473, 4,817,625 and 4,834,109.

Such a respiratory inductance plethysmography uses two separate windings of wire, stitched within an elastic band which is about 10 cm broad, positioned just under the armpits and at the level of the umbilicus. The wires of the two bands, which are equivalent to as many coils, display a self-inductance that varies according to the surface thereof, which in turn depends on the volume variations of the thoraco-abdominal compartment (ΔV_{cw}); in this manner, the volume variations of the thoraco-abdominal compartment (ΔV_{cw}) may be traced by traditional electronic means.

A third example of thoraco-abdominal plethysmography is described by patent GB2116725A, which discloses a thoraco-abdominal plethysmograph integrated in clothing; in this case, the measurement of the volume is obtained by the measurement of appropriate resistances.

Scientific papers in this respect are:
E. R. Post and M. Orth, “Smart Fabric, or Washable Computing”, Proceedings of First International Symposium on Wearable Computers, Cambridge, Mass., IEEE Computer Society, Los Alamitos, Calif. (1997), pp. 167-168;
Della Santa A, Mazzoldi A, De Rossi D. “Dressware: wearable hardware”. Material Sci Eng 1999; C7: 31-37;
Di Rienzo M, Rizzo F, Parati G, Brambilla G, Ferratini M, Castiglioni P. MagIC System: “a New Textile-Based Wearable Device for Biological Signal Monitoring. Applicability in Daily Life and Clinical Setting”. In Proc. IEEE EMB Conference 2005, Shanghai. IEEE Press.

It is the object of the present invention to obtain an apparatus that measures the blood flow between the trunk and the extremities in a non-invasive manner.

According to the invention, such an object is achieved by means of an apparatus for measuring the blood flow between the trunk and the extremities (V_{b}), which comprises a whole body plethysmograph for measuring ΔVL, a thoraco-abdominal plethysmograph for measuring the volume variations of the trunk (ΔV_{cw}) and processing means to analyze said ΔVL with said ΔV_{cw} in order to measure the blood flow between the trunk and the extremities V_{b}, as the difference between ΔV_{cw} and ΔVL:

V_{b}=ΔV_{cw}−ΔVL.

The invention also relates to a process for measuring the blood flow between the trunk and the body periphery (V_{b}), comprising:
a measurement of the volume variations of the trunk (ΔV_{cw}) by means of thoraco-abdominal plethysmography;
a measurement of the variations of gas volume in the lungs (ΔVL) by whole body plethysmography concurrent to the above said thoraco-abdominal plethysmography;
a processing of the volume variations of the trunk (ΔV_{cw}) and of the volume variations of the lungs so as to measure the blood flow between the trunk and the extremities (V_{b}).

By means of an apparatus or a process according to the invention, the blood flow from the trunk to the extremities may be derived from the equation

V_{b}=ΔV_{cw}−ΔVL.

Furthermore, by knowing the instantaneous values of the volume variations of the trunk and the values of the variations of gas volume in the lungs, the instantaneous flow rates (indicated by the symbol P(t)) of the blood flowing from the trunk to the extremities and vice versa, may be derived from the formula

$$P ( t ) = \frac{d {V}_{b}}{d T} = \frac{d ( {ΔV}_{cw} - Δ VL )}{d t} .$$

These and other features of the present invention will become more apparent from the following detailed description of an embodiment thereof by way of no limitation in the accompanying drawings, in which:

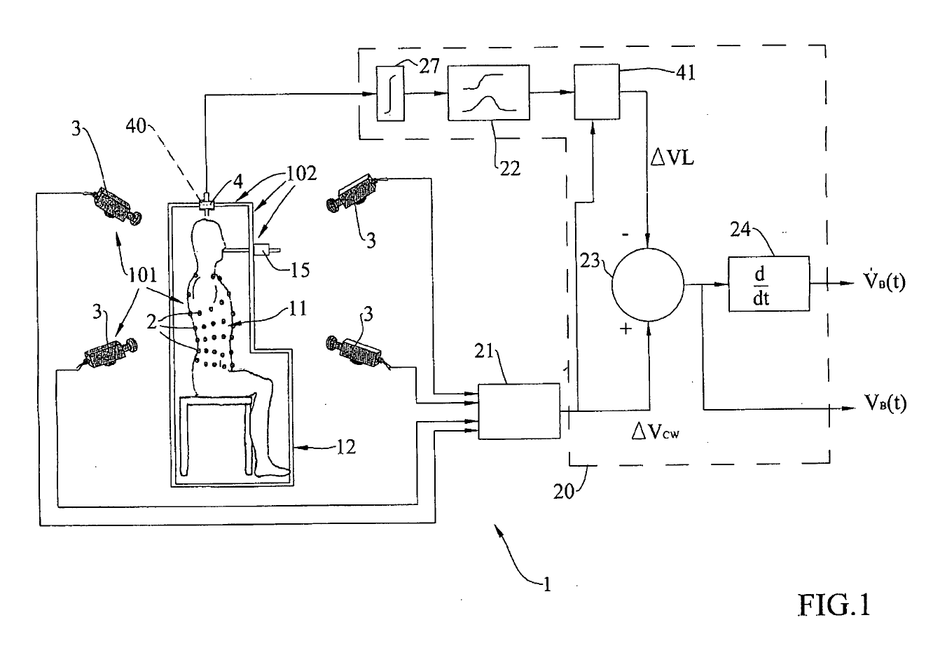

FIG. 1 shows a diagrammatic view of the apparatus in FIG. 1;

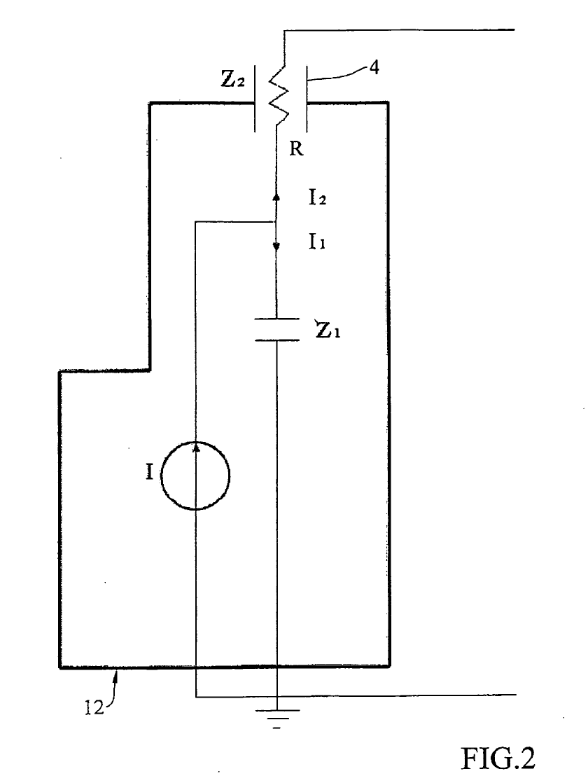

FIG. 2 shows an electric diagram equivalent to the dynamic state of the booth of the plethysmograph in FIG. 1;

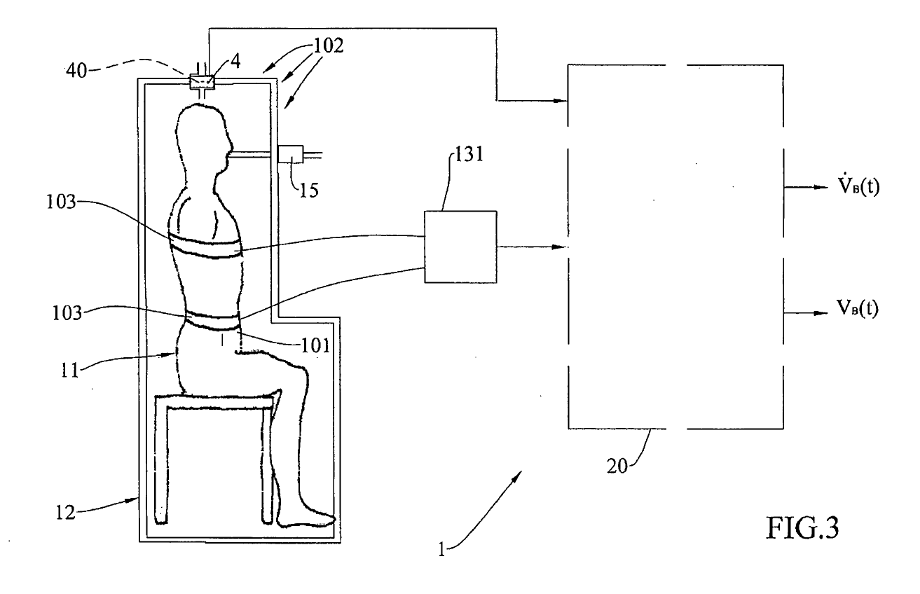

FIG. 3 shows a view similar to that in FIG. 1 for a different embodiment of the invention;

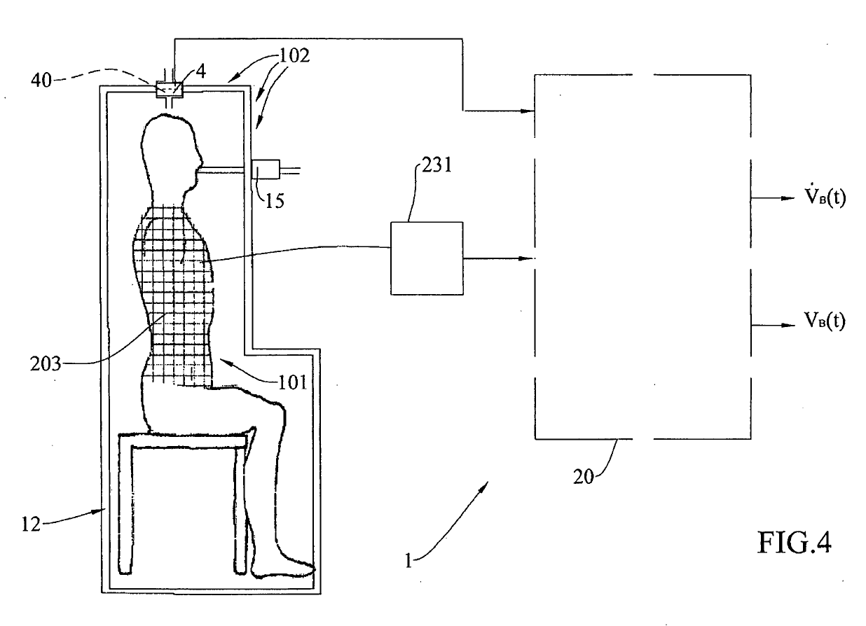

FIG. 4 shows a view similar to that in FIG. 1 for a further embodiment of the invention;

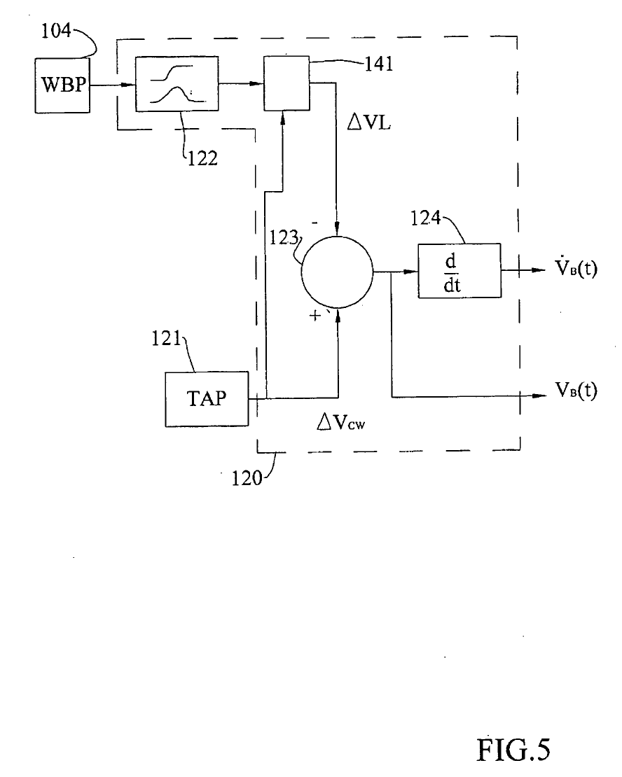

FIG. 5 shows a block diagram of a procedure according to the invention.

An apparatus 1 according to the invention, diagrammatically shown in FIG. 1, is based on the following devices:
a thoraco-abdominal plethysmograph 101, in this case consisting of an opto-electronic plethysmograph, provided with digital video cameras 3 and markers 2;
a whole body plethysmograph 102, by way of example of the flow-type, provided with a booth 12 with a mouthpiece 15, openings 4 and a flow sensor 40;
a microprocessor processing system 20.

The whole body plethysmograph 102 is structured as follows. The booth 12 has a volume of 600 dm³ and is adapted to allow the seating of a subject 11 whose blood flow dynamics between the trunk and the extremities is to be studied. The booth 12 comprises non-transparent parts and transparent parts. Approximately at the level of the mouth of the subject 11, a mouthpiece 15 allows the passage of air from the outside to the inside.

On the upper wall of the booth 12, openings 4 allow the passage of air between the outside and the inside of said booth 12; a traditional flow sensor 40 with a pneumotachograph (provided with Lilly or Silverman or Fleisch resistors) to measure the air flow passing through the openings 4 is applied to each of said openings 4.

The opto-electronic thoraco-abdominal plethysmograph 101 is structured as follows. In front of the booth 12, video cameras 3 are arranged so as to film inside the booth. Such video cameras 3 are connected with an output to an electronic detection device 21.

A plurality of markers 2, consisting of small plastic half-spheres covered by reflective material is applied to the subject 11 at preset positions on the surface of the trunk. The application of such markers 2 is performed by means of the application of double-sided hypoallergenic paper tape.

Said electronic detection device 21 is an electronic system which provides the value of the volume variation of the trunk (ΔV_{cw}) on the outside on the basis of processing performed on the digital images obtained from said video cameras 3 by means of the tracing of the positions in space of the markers 2.

The microprocessor system 20 comprises an integrating circuit 27, an anti-filter 41, an operation block 23 and a differentiator circuit 24.

The flow sensor 40 provides the value of the gas flow in and out of the lungs. Such a value is integrated by the integrating circuit 27 in order to obtain the changes in gas volume of the lungs.

Such a value is anti-filtered by the anti-filter 22 (the explanation will be carried out hereafter).

Downstream of the anti-filter 22 there is a clamping circuit 41. The reason for the use of such a clamping circuit 41 will be explained hereafter.

The values of the outputs of the electronic detection device 21 and clamping circuit 41 are subtracted in the operation block 23 in order to obtain the value of the amount of blood flowing between the trunk and the extremities (V_{b}) and is provided on the outside.

Finally, such a value V_{b} is provided on the outside, and also differentiated by the differentiator circuit 24 to compute the flow rate P(t) thereof.

The reason for the use of the anti-filter 22 is explained hereafter. The dynamical phenomena occurring within the booth 12 must indeed be taken into account. For this purpose, it is helpful to refer to an equivalent circuit shown in FIG. 2, in which:
a current generator (I) represents the variations of lung volume generated by the subject under examination, which partially causes air flow through the walls of the booth and partially compresses the volume of gas contained in the booth during inspiration and decompresses the gas during expiration,
a capacitance C represents the compressibility of the gas contained in the booth and its value depends on the air volume surrounding the subject 11 (equivalent to the volume of the booth subtracted of the total body volume of the subject). An impedance

$${Z}_{1} = \frac{1}{sC}$$

is associated to the capacitance C,
a resistance R (and impedance Z₂=R; such a resistance is valid when the above said Lilly or Silverman resistors are used; when the above said Fleisch resistors are used, the inductance must also be included) represents the pneumotachograph 4;
a current I₂ represents the gas flow through the pneumotachograph 4, and results being correlated to I by the following transfer function:

$${I}_{2} = \frac{1}{1 + sRC} I ,$$

which is the equation of a low pass filter having a cut-off frequency f_{b} equivalent to

$${f}_{b} = \frac{1}{2 π RC}$$

To correct the attenuation and the phase shift introduced by the booth dynamics it is therefore necessary to apply an appropriate anti-filter consisting of a filter, the transfer function of which is:

$$H ( s ) = \frac{1 + s \cdot {τ}_{fb}}{1 + s \cdot {τ}_{fh}}$$

$$where$$

$${τ}_{fb} = \frac{1}{2 π \cdot {f}_{b}}$$

$$and$$

$${τ}_{fh} = \frac{1}{2 π \cdot {f}_{h}}$$

with cut-off frequency fₕ at high frequencies being for instance 20 Hz.

The reason for the use of the clamping circuit 41 is now explained. Within the booth 12, the subject 11 produces heat giving rise to a thermal drift which must be corrected by means of high pass filtering of the signal, which however may be difficult to define. A correction of the drift is therefore obtained by aligning the ΔV_{cw} plots (which are not subject to drift) from the output of the electronic detection device 21 with the output of the anti-filter 22 at the end of the inspiration (when there is usually no blood flow and therefore the two plots coincide) breath by breath and by linearly correcting the signal ΔVL in the segment between the two points of zero flow at the beginning and end of the inspirations. Such a function is performed by the clamping circuit 41.

There may be variants to the embodiment of the present invention.

For instance, as shown in FIG. 3, the thoraco-abdominal plethysmograph 101 could be formed by an above mentioned respiratory inductance plethysmograph (RIP) instead of an opto-electronic plethysmograph. It should be noted that the above mentioned elastic bands 103 comprising electrical coils are used instead of the video cameras 3. The apparatus uses an electronic detection device 131 allowing measurement of the self inductance of said electric coils, providing the value of the volume changes of the trunk to the microprocessor system 20.

According to a further variant, shown in FIG. 4, the thoraco-abdominal plethysmograph 101 could be formed by the aforementioned thoraco-abdominal plethysmograph contained in a shirt 203; the output is read by an electronic detection device 231. For instance, a product designated as Respitrace produced by Vivometrics could be used.

In the two variants just described above, the booth 12 does not need to be transparent, as the use of video cameras is not necessary.

Variants may also be used for the whole body plethysmograph 102, by using, for instance, a volume displacement plethysmograph instead of a variable flow plethysmograph.

FIG. 5 shows a process according to the invention, comprising:
a measurement 104 of the variations of gas volume of the lungs (ΔVL) by means of a whole body plethysmograph.
a measurement 121 of the volume variations of the trunk (ΔV_{cw}) by means of thoraco-abdominal plethysmography; such a measurement 121 is concurrent with the above said measurement 104; said measurement 121 is performed by means of a thoraco-abdominal plethysmograph (which may be, without distinction, an opto-electronic plethysmograph, a respiratory inductance plethysmograph or a plethysmograph contained in clothing,);
a subsequent processing 120 of the volume variations of the trunk (ΔV_{cw}) and of the variations of gas volume of the lungs in order to measure the blood flow between the thoraco-abdominal compartment and the extremities (V_{b}).

The latter processing 120 comprises:
an anti-filtering 122 of the variations of gas volume of the lungs (ΔVL);
a drift correction 141 downstream of the anti-filtering 122;
a computation 123 of the difference between the volume variations of the trunk (ΔV_{cw}) and the volume variations of the lungs (ΔVL) to obtain the volume of blood shifted between the trunk and the extremities (V_{b});

differentiation 124 of V_{b} to obtain the flow of blood P(t) between the trunk and the extremities

1-13. (canceled)

14. An apparatus (1) for measuring the blood flow between the trunk and the extremities (V_{b}), comprising a whole body plethysmograph (102; 4, 12, 15, 22, 40) for measuring the variations of gas volume of the lungs (ΔVL), a thoraco-abdominal plethysmograph (101; 2, 3, 21, 103, 203) for measuring the volume variations of the trunk (ΔV_{cw}) and processing system (20, 21, 22, 23, 24, 41,131, 231) to measure said variations of gas volume of the lungs (ΔVL) with said volume variations of trunk (Δ_{cw}) so as to compute the blood shifts between the trunk and the extremities (V_{b}), said processing system comprising electronic detection devices (21, 131, 231) adapted to measure said volume variations of the trunk (ΔV_{cw}) and a clamping insult (41) adapted to align the plot of said variations of gas volume of the lungs (ΔVL) with the plot of said volume variations of the trunk (ΔV_{cw}) at the end of the inspiration breath by breath and by linearly correcting the variations of gas volume of the lungs (ΔVL) in the segment between two successive points of zero flow at the end of the inspirations.

15. An apparatus according to claim 14, characterised by said thoraco-abdominal plethysmograph (101) is an opto-electronic plethysmograph comprising digital video cameras (3) adapted to film reflective markers (2) placed on the chest and abdomen of a subject being examined (11) seated in a transparent booth (12), with an electronic detection device (21) being provided as connected with an input to said digital video cameras (3), said electronic detection device (21) allowing measurement of the volume variation of the trunk (ΔV_{cx}) on the basis of processing performed on the digital images obtained from said video cameras (3) by means of the tracing of the positions in space of said markers.

16. An apparatus according to claim 14, in which said thoraco-abdominal plethysmograph (101) is a respiratory inductance plethysmograph comprising elastic bands (103) provided with wires, with an electronic detection device (131) being provided, allowing the measurement of the volume variation of the trunk (ΔV_{cx}) on the basis of electric parameters of said wires.

17. An apparatus according to claim 14, in which said thoraco-abdominal plethysmograph (101) is a respiratory inductance plethyamograph contained in a shirt (203), with an electronic detection device (231) being provided, allowing the measurement of the volume variation of the trunk (ΔV_{cx}).

18. An apparatus according to claim 14, characterised by said whole body variable flow plethysmograph (102) comprising a booth (12) provided with a mouthpiece (15) and openings (4) comprising flow sensors (40).

19. An apparatus according to claim 14, characterised by said volume displacement whole body plethysmograph (102).

20. An apparatus according to claim 14, characterised by an anti-filter (22) with a transfer function
$$H ( s ) = \frac{1 + s \cdot {τ}_{fb}}{1 + s \cdot {τ}_{fh}}$$
$$wherein$$
$${τ}_{fb} = \frac{1}{2 π \cdot {f}_{b}}$$
$$and$$
$${τ}_{fh} = \frac{1}{2 π \cdot {f}_{h}}$$
with fb and fh cut-off frequencies at low and high frequencies.

21. An apparatus according to claim 14, characterised by a differentiator circuit (24) allowing to compute the blood flow between the thoraco-abdominal compartment and the periphery (P(t)).

22. A process for measuring the blood flow between the trunk and the extremities (V_{b}), comprising a measurement (121) of the volume variations of the trunk (ΔV_{cw}) by means of a thoraco-abdominal plethysmography, a measurement (104) of the variations of gas volume of the lungs (ΔVL) by means of whole body plethysmography, and a processing (120) of said volume variations of the trunk (ΔV_{cw}) and of said volume variations of the lungs (ΔVL) so as to measure the volume of blood shifted between the trunk and the extremities (V_{b}), said measurements (10, 121) being concurrent, said processing comprising the alignment of the plot of said variations of gas volume of the lungs (ΔVL) with the plot of said volume variations of the trunk (ΔV_{cw}) at the end of the inspiration breath by breath and the linearly correction of the variations of gas volume of the lungs (ΔVL) in the segment between two successive points of zero flow at the end of the inspirations.

23. A process according to claim 22, characterised by an anti-filtering (122) of said gas volume value in the thoraco-abdominal compartment (ΔVL) by means of an anti-filter (22) with a transfer function
$$H ( s ) = \frac{1 + s \cdot {τ}_{fb}}{1 + s \cdot {τ}_{fh}}$$
$$wherein$$
$${τ}_{fb} = \frac{1}{2 π \cdot {f}_{b}}$$
$$and$$
$${τ}_{fh} = \frac{1}{2 π \cdot {f}_{h}}$$
with fb and fh cut-off frequencies at low and high frequencies.

24. A process according to claim 22, characterised by differentiation (124) of the volume of blood shifted between the trunk and the extremities (V_{b}) allowing computation of the flow rate (P(t)) of blood between the thoraco-abdominal compartment and the periphery.

25. An apparatus according to claim 15, characterised by said whole body variable flow plethysmograph (102) comprising a booth (12) provided with a mouthpiece (15) and openings (4) comprising flow sensors (40).

26. An apparatus according to claim 16, characterised by said whole body variable flow plethysmograph (102) comprising a booth (12) provided with a mouthpiece (15) and openings (4) comprising flow sensors (40).

27. An apparatus according to claim 17, characterised by said whole body variable flow plethysmograph (102) comprising a booth (12) provided with a mouthpiece (15) and openings (4) comprising flow sensors (40).

28. An apparatus according to claim 15, characterised by said volume displacement whole body plethysmograph (102).

29. An apparatus according to claim 16, characterised by said volume displacement whole body plethysmograph (102).

30. An apparatus according to claim 17, characterised by said volume displacement whole body plethysmograph (102).

31. An apparatus according to claim 15, characterised by an anti-filter (22) with a transfer function
$$H ( s ) = \frac{1 + s \cdot {τ}_{fb}}{1 + s \cdot {τ}_{fh}}$$
$$wherein$$
$${τ}_{fb} = \frac{1}{2 π \cdot {f}_{b}}$$
$$and$$
$${τ}_{fh} = \frac{1}{2 π \cdot {f}_{h}}$$
with fb and fh cut-off frequencies at low and high frequencies.

32. An apparatus according to claim 16, characterised by an anti-filter (22) with a transfer function
$$H ( s ) = \frac{1 + s \cdot {τ}_{fb}}{1 + s \cdot {τ}_{fh}}$$
$$wherein$$
$${τ}_{fb} = \frac{1}{2 π \cdot {f}_{b}}$$
$$and$$
$${τ}_{fh} = \frac{1}{2 π \cdot {f}_{h}}$$
with fb and fh cut-off frequencies at low and high frequencies.

33. An apparatus according to claim 17, characterised by an anti-filter (22) with a transfer function
$$H ( s ) = \frac{1 + s \cdot {τ}_{fb}}{1 + s \cdot {τ}_{fh}}$$
$$wherein$$
$${τ}_{fb} = \frac{1}{2 π \cdot {f}_{b}}$$
$$and$$
$${τ}_{fh} = \frac{1}{2 π \cdot {f}_{h}}$$
with fb and fh cut-off frequencies at low and high frequencies.
